(19) United States
(12) Patent Application Publication
(10) Pub. No.: US 2006/0241489 A1
(43) Pub. Date: Oct. 26, 2006

(54) Ultrasonically guided puncturing needle

(76) Inventors: Susumu Hiki, Otawara-shi (JP); Shigemitsu Nakaya, Otawara-shi (JP); Hideki Kosaku, Nasushiobara-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(21) Appl. No.: 11/378,436

(22) Filed: Mar. 20, 2006

(30) Foreign Application Priority Data
Mar. 30, 2005 (JP) 2005-099445

(51) Int. Cl. A61B 8/14

(52) U.S. Cl. 600/471

(57) ABSTRACT

An ultrasonically guided puncturing needle stabbed in a subject being irradiated with an ultrasonic wave, the needle includes a cylindrical needle-like member having concaves and convexes formed on a peripheral surface of the needle-like member to reflect the ultrasonic wave, and a film formed on the peripheral surface on which the concaves and convexes are formed.

### CROSS-REFERENCE TO RELATED APPLICATIONS

This application is based upon and claims the benefit of priority from prior Japanese Patent Application No. 2005-099445, filed Mar. 30, 2005, the entire contents of which are incorporated herein by reference.

### BACKGROUND OF THE INVENTION

1. Field of the Invention

The present invention relates to an ultrasonically guided puncturing needle that is stabbed in a subject being irradiated with ultrasonic waves for diagnosis or treatment.

2. Description of the Related Art

What is called ultrasonically guided paracentesis is known in which an operator subjects a lesion site such as tumor which has been found by ultrasonography to puncturing, aspiration biopsy, or cauterization while checking an ultrasonic image of the lesion site. This technique is known to maximize the amount of scattering of ultrasonic waves when the puncturing angle of a needle is set at 60° with respect to an ultrasonic radiation angle. Thus, when the puncturing angle of the needle is not 60°, the amount of backscattering of ultrasonic waves at the tip of the needle may decrease to prevent the ultrasonic waves from being appropriately received. To obtain clear needle tip echoes, it is thus necessary to set the puncturing angle of the needle as close to 60° as possible.

Thus, when this technique is used, a puncturing guide is used which guides the direction in which the needle is inserted. The puncturing guide is commonly fixed to an ultrasonic probe to set the puncturing angle of the needle at 60° with respect to the ultrasonic irradiation angle.

However, even though the inserting direction of the needle is guided using the puncturing guide, the needle itself may be bent during the puncturing process to prevent the puncturing angle from being maintained at 60° near the lesion site. In other cases, another angle may have to be chosen depending on the positional relationship between the ultrasonic probe and the lesion. In the above case, the amount of backscattering of the ultrasonic wave at the needle tip may decrease to make needle tip echoes unclear.

In recent years, a technique relating to a film has been developed in which a gas is used as a reflection source for ultrasonic waves in order to obtain clear needle tip echoes. The gas provides an acoustic impedance significantly different from that of living bodies and can thus be very effectively used as a reflection source for ultrasonic waves (see, for example, PCT National Publication No. 2001-504101).

However, a problem with the technique described in PCT National Publication No. 2001-504101 is that manufacture of the film is very complicated, thus requiring high manufacture costs.

The present invention provides an ultrasonically guided puncturing needle that enables a safe, reliable technique for ultrasonically guided paracentesis to be realized without the need for special equipment or control.

### BRIEF SUMMARY OF THE INVENTION

An ultrasonically guided puncturing needle according to an aspect of the present invention is configured as described below.

(1) An ultrasonically guided puncturing needle stabbed in a subject being irradiated with an ultrasonic wave, the needle comprising a cylindrical needle-like member having concaves and convexes formed on a peripheral surface of the needle-like member to reflect the ultrasonic wave and a film formed on the peripheral surface on which the concaves and convexes are formed.

(2) The ultrasonically guided puncturing needle set forth in (1), wherein a space which is either a gas layer or a vacuum layer is formed in each of the concaves.

(3) The ultrasonically guided puncturing needle set forth in (2), wherein the concaves and convexes are formed on an outer peripheral surface of the needle-like member, and a distance from an outer surface of the film formed on the outer peripheral surface to the space is equal to or shorter than the wavelength of the ultrasonic wave.

(4) An ultrasonically guided puncturing needle stabbed in a subject being irradiated with an ultrasonic wave, the needle comprising a cylindrical needle-like member having a plurality of holes in a peripheral wall and a film which blocks the plurality of the holes.

(5) The ultrasonically guided puncturing needle set forth in (4), wherein a space which is either a gas layer or a vacuum layer is formed in each of the holes.

(6) The ultrasonically guided puncturing needle set forth in (4), wherein the film is formed on an outer peripheral surface of the needle-like member, and

a distance from an outer surface of the film to the space is equal to or shorter than the wavelength of the ultrasonic wave.

(7) The ultrasonically guided puncturing needle set forth in (4), wherein the film blocks the plurality of the holes from an outside of the needle-like member.

(8) The ultrasonically guided puncturing needle set forth in (4), wherein the film blocks the plurality of the holes from an inside of the needle-like member.

(9) An ultrasonically guided puncturing needle stabbed in a subject being irradiated with an ultrasonic wave, the needle comprising a cylindrical needle-like member having a plurality of concaves on an outer peripheral surface and a film which blocks the plurality of the concaves from an outside of the needle-like member.

(10) An ultrasonically guided puncturing needle stabbed in a subject being irradiated with an ultrasonic wave, the needle comprising a cylindrical needle-like member and at least two films stacked on a peripheral surface of the needle-like member, wherein a space which is either a vacuum layer or a gas layer is formed between the two films.

(11) The ultrasonically guided puncturing needle set forth in (10), wherein a distance from an outer surface of the outermost one of the at least two films to the space is equal to or shorter than the wavelength of the ultrasonic wave.

The present invention can realize a safe, reliable technique for ultrasonically guided paracentesis without the need for special equipment or control.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a use environment for an ultrasonically guided puncturing needle according to a first embodiment of the present invention;

FIG. 2A is a schematic diagram of the ultrasonically guided puncturing needle according to the first embodiment;

FIG. 2B is a sectional view of the ultrasonically guided puncturing needle according to the first embodiment;

FIG. 3 is a conceptual drawing showing that an ultrasonic wave is reflected by an air layer according to the first embodiment;

FIG. 4A is a schematic diagram of an ultrasonically guided puncturing needle according to a second embodiment of the present invention;

FIG. 4B is a sectional view of the ultrasonically guided puncturing needle according to the second embodiment of the present invention;

FIG. 5A is a schematic diagram of the ultrasonically guided puncturing needle according to a third embodiment of the present invention;

FIG. 5B is a sectional view of the ultrasonically guided puncturing needle according to the third embodiment of the present invention;

FIG. 6A is a process diagram showing a process of manufacturing an ultrasonically guided puncturing needle according to the third embodiment;

FIG. 6B is a process diagram showing the process of manufacturing an ultrasonically guided puncturing needle according to the third embodiment; and

FIG. 6C is a process diagram showing the process of manufacturing an ultrasonically guided puncturing needle according to the third embodiment.

### DETAILED DESCRIPTION OF THE INVENTION

A first to third embodiments of the present invention will be described with reference to the drawings.

### First Embodiment

(Use Environment for an Ultrasonically Guided Puncturing Needle 30)

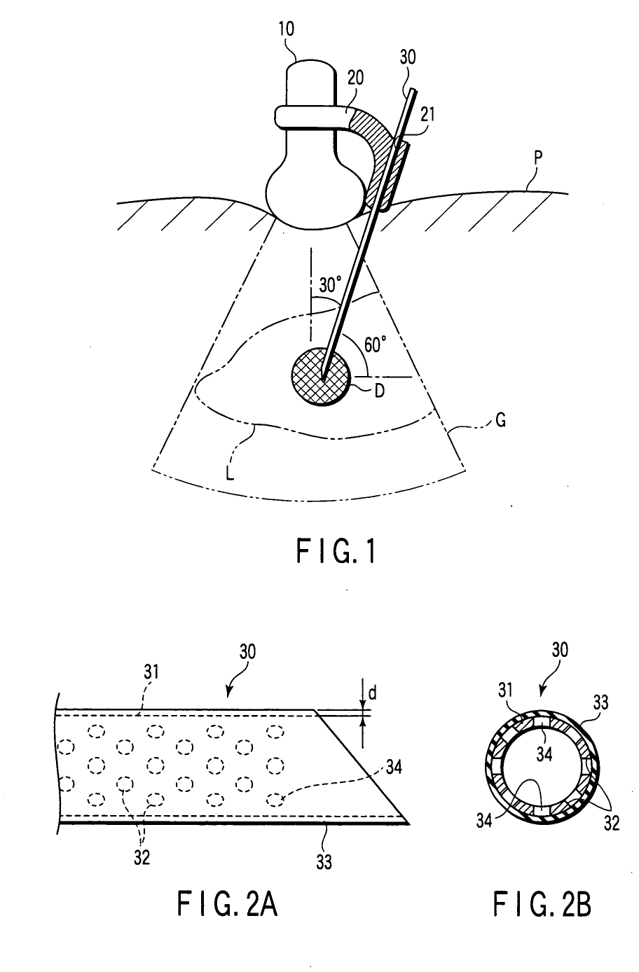

First, a use environment for an ultrasonically guided puncturing needle 30 will be described. FIG. 1 is a schematic diagram showing the use environment for the ultrasonically guided puncturing needle 30 according to the first embodiment of the present invention. In FIG. 1, reference numerals 10, 20, and 30 denote an ultrasonic probe, a puncturing guide, and the ultrasonically guided puncturing needle, respectively. Reference characters P and G denote a subject and an ultrasonic image.

The ultrasonic probe 10 transmits and receives ultrasonic waves through a transmitting and receiving surface provided at a leading end of the ultrasonic probe 10 to the subject P in order to visualize the internal structure of the subject P. An ultrasonic image G of the subject P is displayed on a monitor (not shown). Here, the ultrasonic image G is drawn on the subject P in FIG. 1.

The puncturing guide 20 is fixed to the ultrasonic probe 10 and has a guide hole 21 formed at a predetermined position. The ultrasonically guided puncturing needle 30 is inserted through the guide hole 21 so as to be movable forward and backward. The ultrasonically guided puncturing needle 30 is guided so as to have a fixed puncturing angle. The puncturing angle of the ultrasonically guided puncturing needle 30 is set at about 60°. That is, the ultrasonically guided puncturing needle 30 is stabbed while being inclined at about 30° to the axis of the ultrasonic probe 10 in an array direction. The ultrasonically guided puncturing needle 30 is not inclined in a lens direction.

The ultrasonically guided puncturing needle 30 sucks or cauterizes a biotissue in a lesion site D or inject alcohol into the lesion site D, via its leading end. In the present embodiment, the lesion site D is assumed to be a cancer in the liver L.

(Configuration of the Ultrasonically Guided Puncturing Needle 30)

Now, the configuration of the ultrasonically guided puncturing needle 30 will be described with reference to FIGS. 2A and 2B. FIG. 2A is a schematic diagram of the ultrasonically guided puncturing needle 30 according to the first embodiment. FIG. 2B is a sectional view of the ultrasonically guided puncturing needle 30 according to the first embodiment.

As shown in FIGS. 2A and 2B, the ultrasonically guided puncturing needle 30 comprises a needle main body (needle-like member) 31. The needle main body 31 is formed to be cylindrical and its leading end stabbed in the subject P is reverse-tapered so as to prevent the biotissue from being caught. A metal material is used for the needle main body 31.

A large number of holes 32 are formed in a peripheral wall of the needle main body 31 to allow the inside and outside of the needle main body 31 to communicate with each other. The shape of the hole 32 is not limited but the pitch intervals of the holes are preferably as small as possible. For example, laser machining is used to form holes 32.

A film 33 is formed around an outer peripheral surface of the needle main body 31. The film 33 has a film thickness d equal to or smaller than the wavelength of an ultrasonic wave. The film 33 externally blocks the large number of holes 32 formed in the needle main body 31. This forms a plurality of air layers 34 in the needle main body 31 which are accessible to ultrasonic waves. In order to prevent the needle from being markedly hindered from being inserted into the living body owing to the presence of the holes 32 formed in the needle main body 31, the film 33 is preferably made of resin, which allows a film to be appropriately formed around the needle main body 31 and which is safe for living bodies. The air layers 34 are necessarily formed by the reduced adhesion at the boundary between the hole 32 and the film 33 resulting from the formation of a film 33.

(Usage of the Ultrasonically Guided Puncturing Needle 30)

Now, the usage of the ultrasonically guided puncturing needle 30 will be described. The operator applies the transmitting and receiving surface of the ultrasonic probe 10 to the subject P and starts transmitting and receiving an ultrasonic wave. This causes an ultrasonic image G of a region including the lesion site D to be displayed on the monitor (not shown).

The operator then inserts the ultrasonically guided puncturing needle 30 into the guide hole 21 in the puncturing guide 20. While viewing the ultrasonic image G, the operator stabs the ultrasonically guided puncturing needle 30 in the subject P. The ultrasonically guided puncturing needle 30 stabbed in the subject P is shown in the ultrasonic image G as shown in FIG. 1. Accordingly, while viewing the ultrasonically guided puncturing needle 30 shown in the ultrasonic image G, the operator aligns the leading end of the ultrasonically guided puncturing needle 30 with the lesion site D. The operator then performs an operation such as sucking or cauterization of a biotissue in the lesion site D, injection of alcohol into the lesion site D, or the like. After the operation, the operator removes the ultrasonically guided puncturing needle 30 from the subject P while viewing the ultrasonic image G. The ultrasonically guided paracetesis is thus finished.

(Display of the Ultrasonically Guided Puncturing Needle 30)

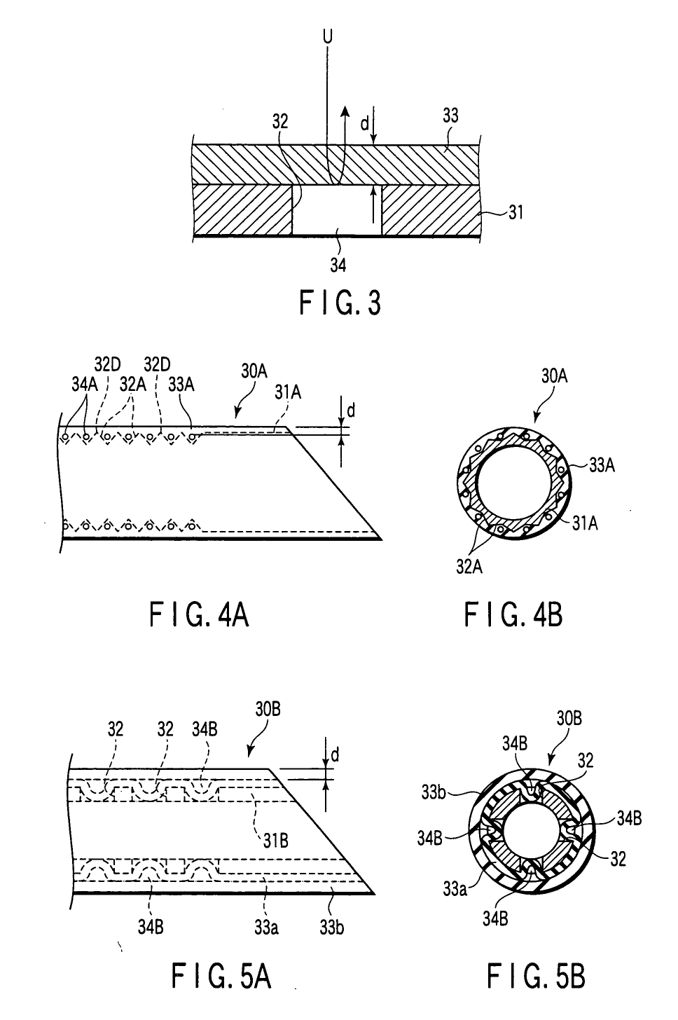

Now, display of the ultrasonically guided puncturing needle 30 will be described with reference to FIG. 3. FIG. 3 is a conceptual drawing showing that an ultrasonic wave is reflected by the air layer 34 according to the first embodiment. Ultrasonic waves transmitted by the ultrasonic probe 10 pass through a tissue in the subject P to reach the ultrasonically guided puncturing needle 30. An ultrasonic wave U which reached a portion of the film 33 corresponding to the hole 32 is transmitted through the film 33 and reflected by the boundary surface between the film 33 and the air layer 34 as shown in FIG. 3. An ultrasonic wave which reached a portion of the film 33 corresponding to the needle main body 31 is transmitted through the film 33 and reflected by the boundary surface between the film 33 and the needle main body 31. The ultrasonic wave reflected by the air layer 34 or needle main body 31 is transmitted through the film 33 and the tissue in the subject P again and then received by the ultrasonic probe 10.

The air layer 34 and the subject P have greatly different acoustic impedances. The ultrasonic wave reflected by the air layer 34 thus has a very large intensity. Consequently, if the ultrasonically guided puncturing needle 30 comprises the large number of air layers 34 as in the case of the present embodiment, the amount of backscattering at the tip of the ultrasonically guided puncturing needle 30 increases to brightly show the ultrasonically guided puncturing needle 30 on the ultrasonic image G.

(Effects of the Present Embodiment)

In the present embodiment, the large number of holes 32 are formed in the peripheral wall of the needle main body 31. The air layers 34 are also provided in the needle main body 31 by blocking the holes 32 from the outside of the needle main body 31 with the film 33.

This increases the amount of backscattering at the tip of the ultrasonically guided puncturing needle 30. The ultrasonically guided puncturing needle 30 is thus brightly shown even if the puncturing angle of the ultrasonically guided puncturing needle 30 is markedly different from 60°. Safe, reliable operations can also be performed without the need for special equipment or control.

Moreover, the present embodiment only requires the formation of a large number of holes 32 in the needle main body 31 and the formation of a film 33 around the outer peripheral surface of the needle main body 31. The ultrasonically guided puncturing needle according to the present embodiment can be obtained by a very simple manufacture process.

The present embodiment has been described in conjunction with the puncturing angle in the array direction. Even if, for example, the ultrasonically guided puncturing needle 30 is greatly bent in the lens direction during the puncturing process, the amount of backscattering at the tip of the ultrasonically guided puncturing needle 30 increases to enable the ultrasonically guided puncturing needle 30 to be shown more brightly than in the prior art.

### Second Embodiment

(Configuration of an Ultrasonically Guided Puncturing Needle 30A)

First, the configuration of an ultrasonically guided puncturing needle 30A will be described with reference to FIGS. 4A and 4B. FIG. 4A is a schematic diagram of the ultrasonically guided puncturing needle 30A according to a second embodiment of the present invention. FIG. 4B is a sectional view of the ultrasonically guided puncturing needle 30A according to the second embodiment.

As shown in FIGS. 4A and 4B, the ultrasonically guided puncturing needle 30A according to the present embodiment comprises a large number of concaves 32A and convexes 32D in an outer peripheral surface of a needle main body (needle-like member) 31A. The shape of the concave 32A and convex 32D is not limited but the pitch intervals are preferably as small as possible. The concaves 32A and the convexes 32D are formed by, for example, sand blasting. Concaves and convexes on an inner peripheral surface can be formed by rotationally inserting a screw-like machine having an outer diameter equal to the inner diameter of the needle main body 31A into the needle main body 31A.

A film 33A is formed around the outer peripheral surface of the needle main body 31A. The film 33A externally blocks the large number of concaves 32A formed in the outer peripheral surface of the needle main body 31A. A small void is formed inside each concave 32A. The distance d from the surface of the film 33A to the void is set equal to or shorter than the wavelength of ultrasonic waves when by conditions are set for the formation of a film 33A. This forms a large number of air layers 34A in the concaves 32A which consist of the voids and which are reachable by supersonic waves.

(Effects of the Present Embodiment)

In the present embodiment, the large number of concaves 32A are formed around the outer peripheral surface of the needle main body 31A. The air layers 34A are provided in the needle main body 31A by blocking the large number of concaves 32A from the outside of the needle main body 31A with the film 33A.

This increases the amount of backscattering at the tip of the ultrasonically guided puncturing needle 30A. The ultrasonically guided puncturing needle 30A is thus brightly shown even if the puncturing angle of the ultrasonically guided puncturing needle 30A is markedly different from 60°. Safe, reliable operations can also be performed without the need for special equipment or control. Moreover, the ultrasonically guided puncturing needle 30A according to the present embodiment can be obtained by a very simple manufacture process.

The present embodiment uses the air layers 34A to increase the amount of backscattering at the tip of the ultrasonically guided puncturing needle 30A. However, the present invention is not limited to this. Any layer, for example, a vacuum layer, may be used provided that it reflects ultrasonic waves well. The vacuum layer is easily obtained provided that a film 33A is formed around the needle main body 31A in a vacuum environment.

### Third Embodiment

(Configuration of an Ultrasonically Guided Puncturing Needle 30B)

First, the configuration of an ultrasonically guided puncturing needle 30B will be described with reference to FIGS. 5A and 5B. FIG. 5A is a schematic diagram of the ultrasonically guided puncturing needle 30B according to a second embodiment of the present invention. FIG. 5B is a sectional view of the ultrasonically guided puncturing needle 30B according to the second embodiment.

As shown in FIGS. 5A and 5B, the ultrasonically guided puncturing needle 30B according to the present embodiment comprises the large number of holes 32 in an outer peripheral surface of a needle main body 31B as in the case of the first embodiment.

A first and second films 33a and 33b are sequentially stacked around the outer peripheral surface of the needle main body 31B. The first film 33a gets into the holes 32, formed in the needle main body 31B, and has concaves formed in its outer peripheral surface at positions corresponding to the holes 32. The second film 33b has a film thickness d equal to or shorter than the wavelength of ultrasonic waves and almost completely cylindrical; the shape of the second film 33b does not coincide with the outer peripheral surface of the first film 33a. This forms a large number of air layers 34B outside the needle main body 31B at positions corresponding to the holes 32; the air layers 34B are blocked by the first and second film 33a and 33b.

(Process of Manufacturing a Ultrasonic Guided Puncturing Needle 30B)

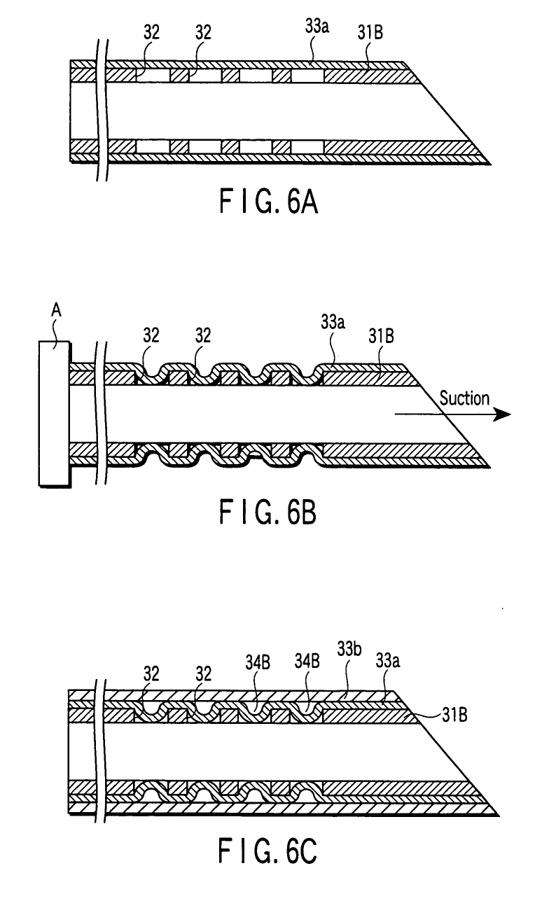

Now, with reference to FIGS. 6A to 6C, description will be given of a process of manufacturing an ultrasonically guided puncturing needle 30B. FIGS. 6A to 6C is a process diagram showing the process of manufacturing an ultrasonically guided puncturing needle 30 30B according to the second embodiment.

As shown in FIG. 6A, a first film 33a is formed around the outer peripheral surface of the needle main body 31B. Then, as shown in FIG. 6B, a base end of the needle main body 31B is closed by a closing member A. Air is sucked from the needle main body 31B through a leading end of the needle main body 31B. This causes the first film 33a to be sucked into the holes 32 to form concaves in the outer peripheral surface of the first film 33a. Then, as shown in FIG. 6C, a second film 33b is formed around the outer peripheral surface of the first film 33a. This forms a large number of air layers 34B around the outer peripheral surface of the needle main body 31B at positions corresponding to the holes 32; the air layers 34B are blocked by the first and second films 33a and 33b.

(Effects of the Present Embodiment)

In the present embodiment, the large number of holes 32 are formed in the peripheral wall of the needle main body 31B. The first and second films 33a and 33b are stacked on the outer peripheral surface of the needle main body 31B. The air layers 34B are provided between the first and second films 33a and 33b to reflect ultrasonic waves.

This increases the amount of backscattering at the tip of the ultrasonically guided puncturing needle 30B. The ultrasonically guided puncturing needle 30B is thus brightly shown even if the puncturing angle of the ultrasonically guided puncturing needle 30A is markedly different from 60°. Safe, reliable operations can also be performed without the need for special equipment or control. Moreover, the ultrasonically guided puncturing needle 30B according to the present invention can be obtained by a very simple manufacture process.

The present embodiment uses the air layers 34B to increase the amount of backscattering at the tip of the ultrasonically guided puncturing needle 30B. However, the present invention is not limited to this. Any layer, for example, a vacuum layer, may be used provided that it reflects ultrasonic waves well. The vacuum layer is easily obtained provided that a second film 33b is formed around the needle main body 31B in a vacuum environment.

The present invention is not limited to the above embodiments proper. In implementation, the components of the embodiments may be varied without departing from the spirit of the present invention. Various inventions can also be formed by appropriately combining a plurality of the components disclosed in the above embodiments. For example, some of the components shown in the embodiments may be deleted. Components of different embodiments may also be appropriately combined together.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonically guided puncturing needle stabbed in a subject being irradiated with an ultrasonic wave, the needle comprising:
a cylindrical needle-like member having concaves and convexes formed on a peripheral surface of the needle-like member to reflect the ultrasonic wave; and
a film formed on the peripheral surface on which the concaves and convexes are formed.

2. The ultrasonically guided puncturing needle according to claim 1, wherein a space which is either a gas layer or a vacuum layer is formed in each of the concaves.

3. The ultrasonically guided puncturing needle according to claim 2, wherein the concaves and convexes are formed on an outer peripheral surface of the needle-like member, and
a distance from an outer surface of the film formed on the outer peripheral surface to the space is equal to or shorter than the wavelength of the ultrasonic wave.

4. An ultrasonically guided puncturing needle stabbed in a subject being irradiated with an ultrasonic wave, the needle comprising:
a cylindrical needle-like member having a plurality of holes in a peripheral wall; and
a film which blocks the plurality of the holes.

5. The ultrasonically guided puncturing needle according to claim 4, wherein a space which is either a gas layer or a vacuum layer is formed in each of the holes.

6. The ultrasonically guided puncturing needle according to claim 5, wherein the film is formed on an outer peripheral surface of the needle-like member, and
a distance from an outer surface of the film to the space is equal to or shorter than the wavelength of the ultrasonic wave.

7. The ultrasonically guided puncturing needle according to claim 4, wherein the film blocks the plurality of the holes from an outside of the needle-like member.

8. The ultrasonically guided puncturing needle according to claim 4, wherein the film blocks the plurality of the holes from an inside of the needle-like member.

9. An ultrasonically guided puncturing needle stabbed in a subject being irradiated with an ultrasonic wave, the needle comprising:
a cylindrical needle-like member having a plurality of concaves on an outer peripheral surface; and
a film which blocks the plurality of the concaves from an outside of the needle-like member.

10. An ultrasonically guided puncturing needle stabbed in a subject being irradiated with an ultrasonic wave, the needle comprising:
a cylindrical needle-like member; and
at least two films stacked on a peripheral surface of the needle-like member,
wherein a space which is either a vacuum layer or a gas layer is formed between the two films.

11. The ultrasonically guided puncturing needle according to claim 10, wherein a distance from an outer surface of the outermost one of the at least two films to the space is equal to or shorter than the wavelength of the ultrasonic wave.
